(19) United States
(12) Patent Application Publication
(10) Pub. No.: US 2015/0283331 A1
(43) Pub. Date: Oct. 8, 2015

(54) TUBE COUPLER CLAMPING AND POSITIONING TOOL

(71) Applicant: Chia-Feng Yu, TAICHUNG (TW)

(72) Inventor: Chia-Feng Yu, TAICHUNG (TW)

(21) Appl. No.: 14/247,834

(22) Filed: Apr. 8, 2014

(51) Int. Cl. A61M 5/32

(52) U.S. Cl. CPC A61M 5/3213

(57) ABSTRACT

A tube coupler clamping and positioning tool comprises a first clamp arm, a second clamp arm and a screw fastening bar. The first and second clamp arms have respectively a first coupling end and a second coupling end that are hinged together. The screw fastening bar has two thread sections formed in different helical directions and runs through the first and second clamp arms with the two thread sections threaded respectively in the first and second clamp arms and the first and second compression ends of the first and second clamp arms opposing each other at a selected interval. Thus through turning the screw fastening bar via a torsional wrench, the interval between the first and second compression ends can be changed to tighten the tube coupler. Moreover, with the torsional wrench, strength of the fastening force and tightening force exerting on the tube coupler also can be controlled.

### FIELD OF THE INVENTION

The present invention relates to a tube coupler and particularly to a tube coupler clamping and positioning tool.

### BACKGROUND OF THE INVENTION

A conventional tube coupler clamping and positioning tool, please refer to FIG. 1, includes two handles 1 hinged together in a cross manner. Each handle 1 has a grip end 2 and a compression end 3. Also referring to FIG. 2, the grip ends 2 can be swiveled towards two sides to open the compression ends 3, and then the two compression ends 3 can clamp a tube coupler 4 to tighten a pliable tube 5 onto a rigid tube 6, namely making the pliable tube 5 be coupled annularly and tightly on the rigid tube 6 without loosening or separating.

The clamping tightness of the pliable tube 5 on the rigid tube 6 through the tube coupler 4 via the conventional tube coupler clamping and positioning tool totally relies on user's feel of hands. Namely, the amount of applying force to make the clamping ends 3 clamp the tube coupler 4 and tighten the pliable tube 5 on the rigid tube 6 totally depends on user's experiences and habits.

Hence, in the event that the force of the tube coupler 4 tightening the pliable tube 5 is too strong and exceeds the structural strength of the rigid tube 6 or tube coupler 4, the rigid tube 6 could be deformed or even fractured to result in leakage of liquid flowing, or the tube coupler 4 could be directly ruptured. On the other hand, in the event that the tightening force is too small, the pliable tube 5 and rigid tube 6 could loosen off and separate to result in liquid leakage.

Thus, depending on user's habits and experiences to control the force of the tube coupler 4 to tighten the pliable tube 5 via the conventional tube coupler clamping and positioning tool is unreliable. Because the tightening force of the tube coupler 4 is affected by human factor and reliability suffers, it cannot fully meet use requirements. There is still room for improvement.

### SUMMARY OF THE INVENTION

Therefore, the primary object of the present invention is to provide a tube coupler clamping and positioning tool to control the tightening force exerting to the pliable tube to avoid too strong or too weak tightening force that may result in fracture of the rigid tube, or rupture or loosening of the tube coupler.

The tube coupler clamping and positioning tool of the invention aims to tighten a tube coupler and couple a pliable tube onto a rigid tube, and comprises a first clamp arm, a second clamp arm and a screw fastening bar. The first clamp arm has a first coupling end and a first compression end. The second clamp arm has a second coupling end and a second compression end. The screw fastening bar has two thread sections formed in different helical directions. The first and second coupling ends are hinged together. The screw fastening bar runs through the first clamp arm and second clamp arm with the two thread sections threaded respectively in the first clamp arm and second clamp arm and the first and second compression ends opposing each other at a selected interval.

Thus, through a torsional wrench, the screw fastening bar can be wrenched to change the distance of the interval, thereby the first and second compression ends can clamp and tighten the tube coupler. With the torsional wrench, the output fastening force also can be controlled as desired to avoid too strong or too weak of the tightening force exerting to the tube coupler, thereby can prevent fracture of the rigid tube, or rupture or loosening of the tube coupler to meet use requirement.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of a conventional tube coupler clamping and positioning tool.

FIG. 2 is a schematic view of a conventional tube coupler clamping and positioning tool in a use condition.

FIG. 3 is a schematic view of the invention.

FIG. 4 is a schematic view of the tube coupler of the invention in a use condition.

FIGS. 5A through 5C are schematic views of the invention in operating conditions.

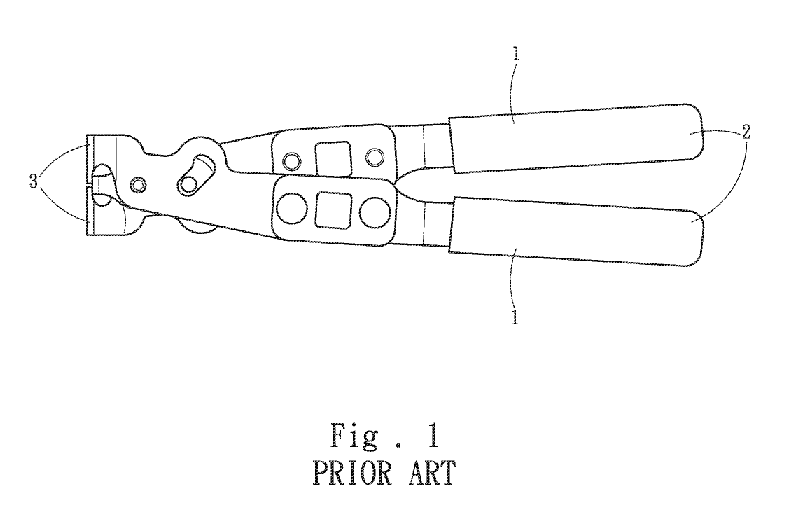

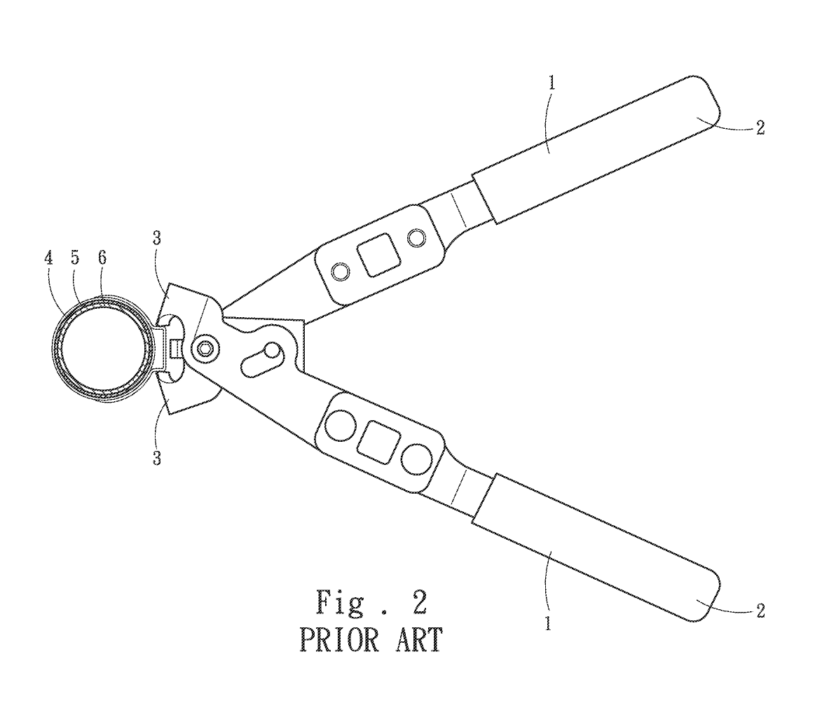

### DETAILED DESCRIPTION OF THE PREFERRED EMBODIMENT

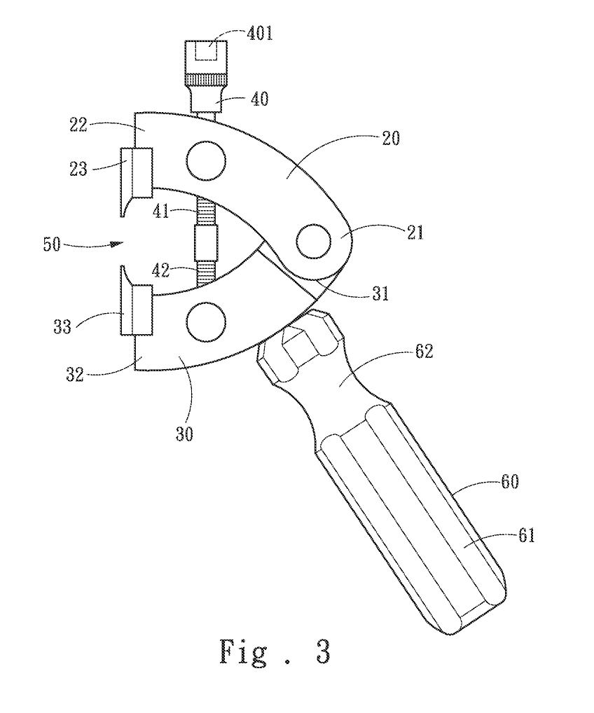

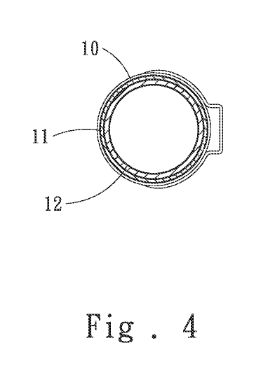

Please refer to FIGS. 3 and 4, the present invention aims to provide a tube coupler clamping and positioning tool to tighten a tube coupler 10 and couple a pliable tube 11 onto a rigid tube 12. The tool according to the invention comprises a first clamp arm 20, a second clamp arm 30 and a screw fastening bar 40. The first clamp arm 20 has a first coupling end 21 and a first compression end 22. The second clamp arm 30 has a second coupling end 31 and a second compression end 32. The first and second compression ends 22 and 32 have respectively a clamp head 23 and 33 to enhance clamp effectiveness and usability.

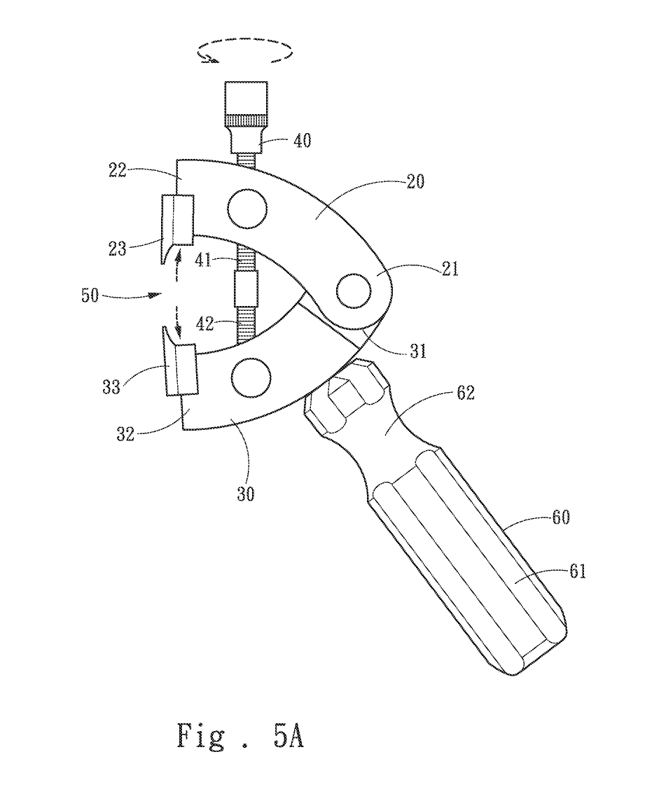

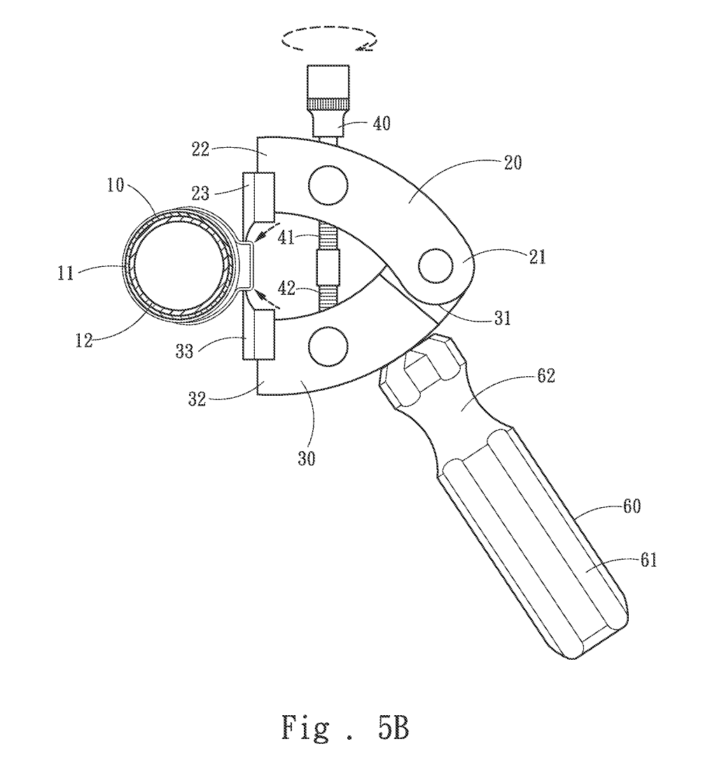

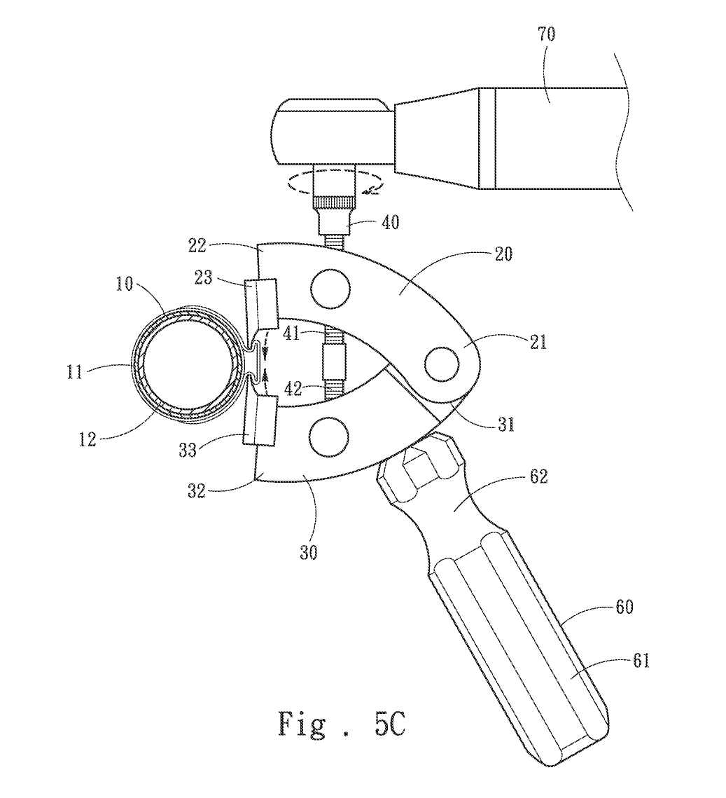

The screw fastening bar 40 has two thread sections 41 and 42 formed in different helical directions, and a coupling cavity 401 at an outer end to be wedged by a general torsional wrench 70 (referring to FIG. 5C) when in use. The first and second coupling ends 21 and 31 are hinged together. The screw fastening bar 40 runs through the first clamp arm 20 and second clamp arm 30 with the two thread sections 41 and 42 threaded respectively in the first clamp arm 20 and second clamp arm 30 and the first and second compression ends 22 and 32 opposing each other at a selected interval 50.

The second clamp arm 30 is fastened to a handgrip 60 which has a plurality of anti-skid flutes 61 formed on the surface to prevent slipping when in use to facilitate user's gripping. The handgrip 60 further has an indented curved surface 62 formed annularly on the surface to further enhance gripping steadiness. Moreover, the handgrip 60 and the screw fastening bar 40 can be located not on the same straight line, namely the handgrip 60 and screw fastening bar 40 are positioned at a skewed angle against each other to improve handling feel of user's hands when in use.

By means of the structure set forth above, through the two thread sections 41 and 42 formed in different helical directions, two fastening forces in different directions can be provided. Hence by wrenching the screw fastening bar 40, the interval 50 can be changed to varying sizes, and the first and second compression ends 22 and 32 of the first and second clamp arms 20 and 30 can tighten the tube coupler 10 as desired. When in use, please refer to FIGS. 5A through 5C, first, wrenching the screw fastening bar 40 to move the first and second compression ends 22 and 32 away from each other until the interval 50 is slightly greater than the tube coupler 10 (as shown in FIG. 5A); next, placing the tube coupler 10 between the first and second compression ends 22 and 32, and then wrenching manually the screw fastening bar 40 to make the first and second compression ends 22 and 32 contact the tube coupler 10 (referring to FIG. 5B); finally, wrenching the screw fastening bar 40 via the torsional wrench 70 to tightly clamp the tube coupler 10 (referring to FIG. 5C). Through the torsional wrench, the output fastening force can be controlled easily to evenly shrink the tube coupler 10 to couple the pliable tube 11 onto the rigid tube 12. As the torsional force value output from the torsional wrench can be obtained clearly, the tightening force exerting to the tube coupler 10 can be controlled not being too strong or too weak, thereby can prevent deformation or fracture of the rigid tube 12, or rupture or loosening of the tube coupler 10.

As a conclusion, by using the torsional wrench to control the output fastening force, the invention allows users to deliver a torsional force as desired, thus can avoid the problem of too strong or too weak tightening force under the operation specification to meet use requirements.

What is claimed is:

1. A tube coupler clamping and positioning tool to tighten a tube coupler and couple a pliable tube onto a rigid tube, comprising:
a first clamp arm including a first coupling end and a first compression end;
a second clamp arm including a second coupling end and a second compression end, the first coupling end and the second coupling end being hinged together; and
a screw fastening bar which includes two thread sections formed in different helical directions and runs through the first clamp arm and the second clamp arm with the two thread sections threaded respectively in the first clamp arm and the second clamp arm and the first compression end and the second compression end opposing each other at a selected interval.

2. The tube coupler clamping and positioning tool of claim 1, wherein the first compression end and the second compression end include respectively a first clamp head and a second clamp head.

3. The tube coupler clamping and positioning tool of claim 1, wherein the second clamp arm is fastened to a handgrip.

4. The tube coupler clamping and positioning tool of claim 3, wherein the handgrip includes a plurality of anti-skid flutes on the surface thereof.

5. The tube coupler clamping and positioning tool of claim 3, wherein the handgrip includes an annular indented curved surface.

6. The tube coupler clamping and positioning tool of claim 3, wherein the handgrip and the screw fastening bar are not positioned on a same straight line.

7. The tube coupler clamping and positioning tool of claim 1, wherein the screw fastening bar includes a coupling cavity at an outer end thereof.
